(19) United States
(12) Patent Application Publication
(10) Pub. No.: US 2004/0087569 A1
(43) Pub. Date: May 6, 2004

(54) N-heterocyclic bicyclic lactone compounds

(76) Inventors: Todd D. Nelson, Exton, PA (US); Carl LeBlond, Johnstown, PA (US); Michael H. Kress, Exton, PA (US)

Correspondence Address: MERCK AND CO INC, P O BOX 2000, RAHWAY, NJ, 070650907

(21) Appl. No.: 10/694,647

(22) Filed: Oct. 28, 2003

Related U.S. Application Data
(60) Provisional application No. 60/422,556, filed on Oct. 31, 2002.

(51) Int. Cl. A61K 31/397; C07D 498/02

(52) U.S. Cl. 514/210.16; 514/210.17; 544/105; 540/560; 548/953

(57) ABSTRACT

Novel N-heterocyclic bicyclic lactone compounds of formula I and its novel hydroxyamide precursors of formula IV,

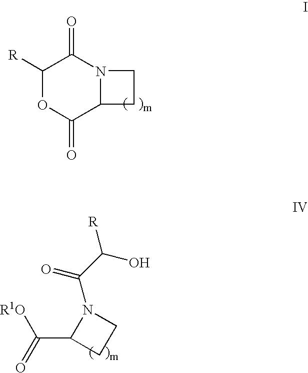

are synthesized by coupling a hydroxy acid of formula II with an ester of formula III or a pharmaceutically acceptable salt thereof,

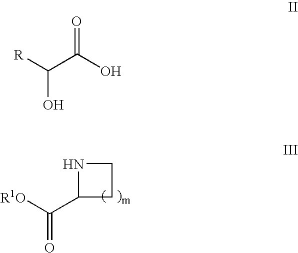

in the presence of a peptide coupling reagent to produce a hydroxyamide of formula IV, and cyclizing the hydroxyamide of formula IV to produce compounds of formula 1.

### CROSS REFERENCE TO RELATED APPLICATIONS

This application claims the benefit of U.S. Provisional Application No. 60/422,556, filed Oct. 31, 2002.

### BACKGROUND OF THE INVENTION

Thrombin is a serine protease present in blood plasma in the form of a precursor, prothrombin. Thrombin plays a central role in the mechanism of blood coagulation by converting the solution plasma protein, fibrinogen, into insoluble fibrin. Thrombin inhibition is useful in treating and preventing a variety of thrombotic conditions, such as coronary artery and cerebrovascular disease, and preventing coagulation of stored whole blood or coagulation in other biological samples for testing or storage. Thrombin inhibitors can be added to or contacted with any medium containing or suspected of containing thrombin and in which inhibiting blood coagulation is desired, e.g., when contacting a mammal's blood with vascular grafts, stents, orthopedic prosthesis, cardiac prosthesis, and extracorporeal circulation systems. Those experienced in this field are readily aware of the circumstances requiring anticoagulant therapy.

Molecules that selectively inhibit the formation of thrombin or modulate the activity of thrombin have the potential to regulate many of the above-mentioned disease states. Proline-derivative thrombin inhibitor 1 is one such molecule. The known synthesis of this compound (see WO 02/50056) requires linear assembly using standard peptide coupling and multiple protection and deprotection manipulations.

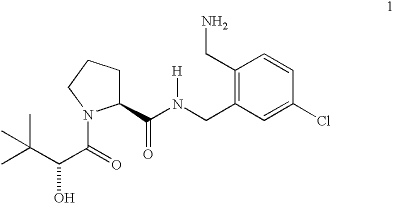

The present invention is an efficient process for preparing compound 1 and structurally related compounds.

### SUMMARY OF THE INVENTION

This invention is directed to synthesis of novel N-heterocyclic bicyclic lactone compounds of formula I and its novel hydroxyamide precursors of formula IV:

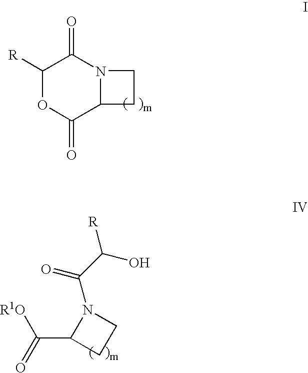

comprising coupling an hydroxy acid of formula II with an ester of formula III:

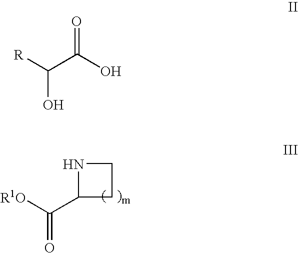

in the presence of a peptide coupling agent to produce the novel hydroxy amide of formula IV, wherein

R is

a) C₁₋₆ alkyl unsubstituted or substituted with one, two, or three groups independently selected from C₆₋₁₀ aryl, C₁₋₆ alkoxy, halogen, and amino; or

b) a 6-10 membered monocyclic or bicyclic aryl ring system, unsubstituted or substituted with one, two or three groups independently selected from C₁₋₆ alkyl, C₁₋₆ alkoxy, halogen, and amino;

R¹ is

a) C₁₋₆ alkyl unsubstituted or substituted with one, two, or three groups independently selected from C₆₋₁₀ aryl, hydroxy, C₁₋₆ alkoxy, halogen, and amino;

b) benzyl unsubstituted or substituted with one, two or three groups independently selected from C₁₋₆ alkyl, hydroxy, C₁₋₆ alkoxy, halogen, and amino; or

c) hydrogen; and

m is 1, 2, 3, 4, or 5.

The novel hydroxyamide of formula IV may be cyclized in the presence of an acid to produce the novel N-heterocyclic bicyclic lactone of formula I.

The invention also includes novel heterocyclic bicyclic lactone compounds of formula I and its novel precursors of formula IV as well as pharmaceutically acceptable derivatives or solvates of such formulas.

The invention also includes methods of using the novel N-heterocyclic bicyclic compounds of formula I and its novel precursors to make thrombin inhibitors by coupling the novel N-heterocyclic bicyclic compounds with

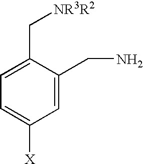

to form a compound of formula V,

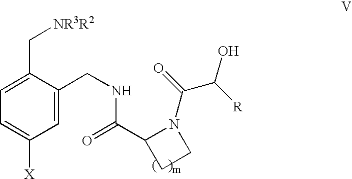

wherein

R is

a) C₁₋₆ alkyl unsubstituted or substituted with one, two, or three groups independently selected from C₆₋₁₀ aryl, C₁₋₆ alkoxy, halogen, and amino; or

b) 6-10 membered monocyclic or bicyclic aryl, unsubstituted or substituted with one, two or three groups independently selected from C₁₋₆ alkyl, C₁₋₆ alkoxy, halogen, and amino group;

R² is an amino protecting group;

R³ is hydrogen or an amino protecting group;

m, is 1, 2, 3, 4, or 5; and

X is a halogen selected from the group consisting of F, Br, I, or Cl.

The foregoing general description and the following detailed description are exemplary and are intended to provide further explanation of the invention claimed.

### DETAILED DESCRIPTION OF THE INVENTION

The invention includes synthesis of novel N-heterocyclic bicyclic lactone compounds and its novel precursors and methods of using such N-heterocyclic bicyclic lactone compounds and precursors to make thrombin inhibitors. An embodiment of the synthesis of novel N-heterocyclic bicyclic lactone compounds, such as formula I, and its novel hydroxy amide precursor of formula IV,

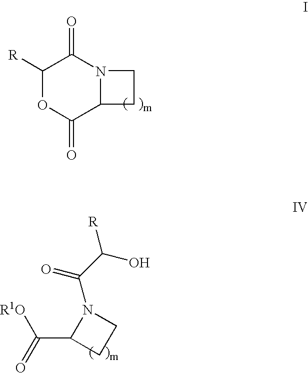

comprises coupling a hydroxy acid of formula II with an ester of formula III or a pharmaceutically acceptable salt thereof,

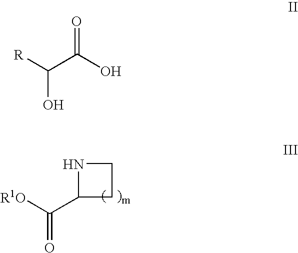

in the presence of a peptide coupling reagent, to produce the novel hydroxyamide of formula IV, where

R is

a) C₁₋₆ alkyl unsubstituted or substituted with one, two, or three groups independently selected from C₆₋₁₀ aryl, C₁₋₆ alkoxy, halogen, and amino; or

b) a 6-10 membered monocyclic or bicyclic aryl ring system, unsubstituted or substituted with one, two or three groups independently selected from C₁₋₆ alkyl, C₁₋₆ alkoxy, halogen, and amino;

R¹ is

a) C₁₋₆ alkyl unsubstituted or substituted with one, two, or three groups independently selected from C₆₋₁₀ aryl, hydroxy, C₁₋₆ alkoxy, halogen, and amino;

b) benzyl unsubstituted or substituted with one, two or three groups independently selected from C₁₋₆ alkyl, hydroxy, C₁₋₆ alkoxy, halogen, and amino; or

c) hydrogen; and

m is 1, 2, 3, 4, or 5.

The novel hydroxyamide of formula IV may be cyclized in the presence of an acid to produce the novel N-heterocyclic bicyclic lactone of formula I.

In one embodiment, R is C 1-6 alkyl, e.g. tert-butyl. In another embodiment, m is 1. In another embodiment, R1 is methyl. In another embodiment, the peptide coupling reagent is 1-ethyl-3-(3-dimethylaminopropyl)carbodiimide hydrochloride. In another embodiment, the acid is toluene sulfonic acid.

The invention also includes novel N-heterocyclic bicyclic lactone compounds of formula I and its novel hydroxyamide precursors of formula IV as well as pharmaceutically acceptable derivatives or solvates of the general formulas:

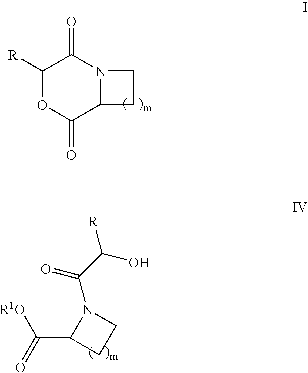

wherein

R is

a) C₁₋₆ alkyl unsubstituted or substituted with one, two, or three groups independently selected from C₆₋₁₀ aryl, C₁₋₆ alkoxy, halogen, and amino; or

b) a 6-10 membered monocyclic or bicyclic aryl ring system, unsubstituted or substituted with one, two or three groups independently selected from C₁₋₆ alkyl, C₁₋₆ alkoxy, halogen, and amino;

R¹ is

a) C₁₋₆ alkyl unsubstituted or substituted with one, two, or three groups independently selected from C₆₋₁₀ aryl, hydroxy, C₁₋₆ alkoxy, halogen, and amino;

b) benzyl unsubstituted or substituted with one, two or three groups independently selected from C₁₋₆ alkyl, hydroxy, C₁₋₆ alkoxy, halogen, and amino; or

c) hydrogen; and

m is 1, 2, 3, 4, or 5.

In one embodiment, R is C 1-6 alkyl, e.g. tert-butyl. In another embodiment, m is 1. In another embodiment, R1 is methyl.

The invention also includes methods of using these novel N-heterocyclic bicyclic compounds of formula I and its novel precursors to make thrombin inhibitors by coupling the novel N-heterocyclic bicyclic compounds with

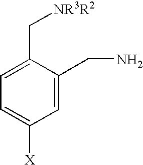

to form a compound of formula V,

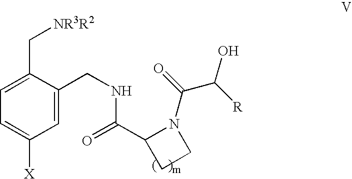

wherein

R is

a) C₁₋₆ alkyl unsubstituted or substituted with one, two, or three groups independently selected from C₆₋₁₀ aryl, C₁₋₆ alkoxy, halogen, and amino; or

b) 6-10 membered monocyclic or bicyclic aryl, unsubstituted or substituted with one, two or three groups independently selected from C₁₋₆ alkyl, C₁₋₆ alkoxy, halogen, and amino group;

R² is an amino protecting group;

R³ is hydrogen or an amino protecting group;

m, is 1, 2, 3, 4, or 5; and

X is a halogen selected from the group consisting of F, Br, I, or Cl.

In one embodiment, R² is tert butoxy carbonyl or carbobenzoxy. In another embodiment, R is C 1-6 alkyl, e.g. tert butyl. In another embodiment, the solvent is a polar solvent selected from the group of consisting of triethylamine, isopropyl alcohol, N-methyl pyrrolidinone, dimethylformamide, diisopropyethylamine, CH₃CN, and tetrahydrofuran. In another embodiment, R³ is hydrogen.

Compounds of formula (I)-(V) may have chiral centers and occur as racemic mixtures, as individual diastereomers, or as enantiomers with all isomeric forms. The scope of the present invention includes individual enantiomers of compounds of formula (I)-(V) as well as mixtures of enantiomers of compounds of formula (I)-(V) in any proportion, including racemic mixtures. Generally it is preferred to use a compound of formula (I)-(V) in the form of a purified single enantiomer, most preferably the (S) isomer.

When any variable occurs more than one time in any constituent or in formula I-V, its definition on each occurrence is independent of its definition at every other occurrence. Also, combinations of substituents and/or variables are permissible only if such combinations result in stable compounds.

Compounds prepared according to the process of the invention are useful in preparing compounds that are useful for treating or preventing a variety of thrombotic conditions, including venous thromboembolism (e.g. obstruction or occlusion of a vein by a detached thrombus, obstruction or occlusion of a lung artery by a detached thrombus), cardiogenic thromboembolism (e.g. obstruction or occlusion of the heart by a detached thrombus), arterial thrombosis (e.g. formation of a thrombus within an artery that may cause infarction of tissue supplied by the artery), atherosclerosis (e.g. arteriosclerosis characterized by irregularly distributed lipid deposits) in mammals, and lowering the propensity of devices that come into contact with blood to clot blood.

Some abbreviations that may appear in this application are as follows:

### Abbreviations

Designation

| | |
|---|---|
| ACN | acetonitrile |
| Boc | tert-butoxycarbonyl |
| DBU | 1,8-diazabicyclo[5.4.0]undec-7-ene |
| CH₃CN | acetonitrile |
| DCM | dichloromethane |
| DIEA | diisopropylethylamine |
| DIPEA | diisopropyethylamine |
| DMF | dimethylformamide |
| EDC | 1-ethyl-3-(3-dimethylaminopropyl)carbodiimide hydrochloride |
| EtOH | ethanol |
| HOBt | 1-hydroxybenzotriazole hydrate |
| IPAc | isopropyl acetate |
| IPA | isopropyl alcohol |
| MeOH | methanol |
| NaBH₄ | sodium borohydride |
| Na₂CO₃ | sodium carbonate |
| NMP | N-methyl pyrrolidinone |
| PTSA | p-toluenesulfonic acid |
| TEA | triethylamine |
| TFA | trifluoroacetic acid |
| THF | tetrahydrofuran |

Unless otherwise noted, the term “alkyl” includes both branched- and straight chain saturated aliphatic hydrocarbon groups having the specified number of carbon atoms for example, “C₁₋₆ alkyl” means an alkyl group having 1 to 6 carbon atoms, e.g., 1, 2, 3, 4, 5 or 6.” For illustration and not limitation, the alkyl may be methyl, ethyl, propyl, butyl, etc. The alkyl group may be unsubstituted or substituted with, for example, C₆₋₁₀ aryl, hydroxy, C₁₋₆ alkoxy, halogen, or amino.

Unless otherwise noted, “halogen”, as used herein,includes fluorine, chlorine, bromine, and iodine.

Unless otherwise noted, “alkoxy” means a linear or branched alkyl group of indicated number of carbon atoms attached through an oxygen bridge. “C₁₋₆ alkoxy” means any alkoxy having 1 to 6 carbon atoms, e.g., 1, 2, 3, 4, 5 or 6.

Unless otherwise noted, the term “aryl” includes a “C₁₋₆ alkoxy” means on alkoxy group having 6- to 10-membered mono- or bicyclic ring system such as phenyl, or naphthyl. The aryl ring can be unsubstituted or substituted with, for illustration and not limitation, one or more of C₁₋₆ alkyl; hydroxy; C₁₋₆ alkoxy; halogen; or amino.

Unless otherwise noted, the term “solvent” includes any polar solvent such as, for example, triethylamine, isopropyl alcohol, N-methyl pyrrolidinone, dimethylformamide, diisopropyethylamine, CH₃CN and tetrahydrofuran.

Unless otherwise noted, the term “acid” includes any acid such as a Bronsted Lawry or Lewis acid donating a proton or receiving an electron, which has a pH lower than 7.

Unless otherwise noted, the term “amino protecting group” includes, for illustration and not limitation, —C(O)OR, wherein R is any alkyl group such as C₁₋₄ alkyl.

Unless otherwise noted, “peptide coupling reagent” includes any class of compounds that mediate the coupling of an amine and a carboxylic acid such as, for example, 1-ethyl-3-(3-dimethylaminopropyl) carbodiimide hydrochloride.

For illustration and not limitation, an example of the novel hydroxyamide of general formula IV for use according to the invention includes:

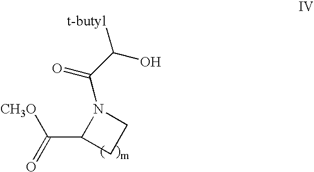

and pharmaceutically acceptable derivatives or solvates thereof. The compound of formula (IV) may be in the form of a purified single enantiomer, (S) or (R) isomer, or a mixture of both. For illustration and not limitation, the hydroxyamide of general formula IV has been described with R as t-butyl and R1 as CH3. The R and R1 groups of general formula IV may be any designated R and R1 groups, respectively, independent of each other. For example, when R is t-butyl, R1 may be an alkyl, including methyl, substituted or unsubstituted benzyl, or hydrogen. Similarly, when R1 is a methyl, R may be an alkyl, including tert butyl, or an aryl group.

For illustration and not limitation, an example of the novel N-heterocyclic bicyclic lactone of general formula I for use according to the invention include:

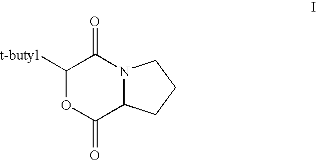

and pharmaceutically acceptable derivatives or solvates thereof. The compound of formula (I) may be in the form of a purified single enantiomer, (S) or (R) isomer, or a mixture of both. For illustration and not limitation, the N-heterocyclic bicyclic lactone compound of general formula I has been described with R as t-butyl. The R group of the N-heterocyclic bicyclic lactone compound of general formula I may be any other designated R group. For example, R may be an alkyl other than tert butyl or an aryl group.

It will be appreciated by those skilled in the art that the compounds of formula I-V may be modified to provide pharmaceutically acceptable derivatives thereof at any of the functional groups in the compounds of formula I-V. Such derivatives are clear to those skilled in the art, without undue experimentation.

The pharmaceutically-acceptable salts of the compounds of the invention prepared according to the procedures described herein include those derived from pharmaceutically acceptable inorganic and organic acids such as e.g. hydrochloric, hydrobromoic, sulfuric, sulfamic, phosphoric, nitric and the like, or the quaternary ammonium salts which are formed, e.g., from inorganic or organic acids or bases. Examples of acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, nitrate, oxalate, pamoate, pectinate, persulfate, 3-phenylpropionate, picrate, pivalate, propionate, succinate, sulfate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

General Scheme I demonstrates, for illustration and not limitation, a synthesis of novel N-heterocyclic bicyclic lactones of formula I and its novel hydroxyamide precursors of formula IV.

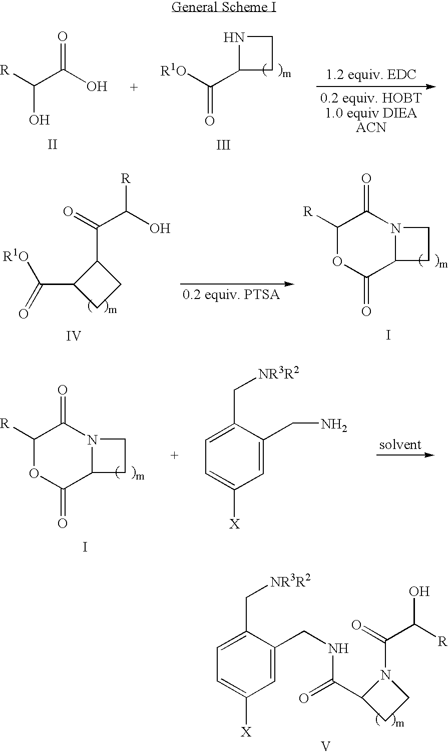

All variables are as previously defined.

The inventive process comprises coupling a hydroxy acid of formula II, such as (R)-5, with an ester of formula III attached to an N based heteroalkyl ring, such as proline. Various hydroxy acids such as, for illustration and not limitation, (R)-3,3-dimethyl-2-hydroxybutyric acid (R)-5 may be used. Although (R)-3,3-dimethyl-2-hydroxybutyric acid is a relatively simple chiral hydroxy acid, and these types of molecules are commonly used as synthetic building blocks, only sparse reports on the synthesis of either antipode of this molecule exist. Coppola, G. M.; Schuster, H. F., I-Hydroxy Acids in Enantioselective Syntheses; VCH, Weinheim 1997. For example: A classical resolution with cinchonidine has been reported, but is low yielding and requires multiple crystallizations: Tanabe, T.; Yajima, S.; Imaida, M. Bull. CHEM. SOC. JPN. 1968, 41, 2178. The conversion of 3,3-dimethyl-2-oxobutanoic acid 2 to (R)-2-hydroxy-3,3-dimethylbutancarboxylic acid has been reported to proceed with a high level of enantioselectivity using the cell line Proteus vulgaris, H₂ gas and benzylviologen. Schummer, A.; Yu, H.; Simon, H. TETRAHEDRON 1991, 47, 9019; Simon, H.; Bader, J.; Günther, H.; Neumann, S.; Thanos, J. Angew. CHEM. INT. ED. ENGL. 1985, 24,539. The synthesis of the (S)2-hydroxy-3,3-dimethylbutancarboxylic acid has been accomplished by diazotization of (L)-tert-leucine. Quast, H.; Leybach, H. CHEM. BER. 1991, 124, 849; Van Draanen, N. A., Arseniyadis, S.; Crimmins, M. T.; Heathcock, C. H. J. Org. Chem. 1991, 56, 2499; Hartwig, W.; Schoellkopf, U. LIEBIGS ANN. CHEM. 1982, 1952. This material can also be obtained from the racemate by a classical resolution with either brucine (ref. 10) or (S)-phenylethylamine. Zhang, W.-Y.; Jakiela, D. J.; Maul, A.; Knors, C.; Lauher, J. W.; Helquist, P.; Enders, D. J. AM. CHEM. Soc. 1988, 110, 4652; Masamune, S.; Reed, L. A. III, Davis, J. T.; Choy, W. J. ORG. CHEM. 1983, 48, 4441. Similarly, the asymmetric reduction of methyl 3,3-dimethyl-2-oxobutanoate using stoichiometric amounts of borohydrides has been used to from the corresponding (S)-carbinol. Brown, H. C.; Cho, B. T.; Park, W. S. J. ORG. CHEM. 1986, 51. 3396; Brown, H. Pai, G. G. J. ORG. CHEM. 1985, 50, 1384. This material has also been prepared by a multistep synthesis: Ko, K.-Y.; Frazee, W. J.; Eliel, E. L. TETRAHEDRON 1984, 40, 1333.

The following examples are for illustration and not limitation.

### EXAMPLE 1

An embodiment, for illustration and not limitation, of the synthesis of novel N-heterocyclic bicyclic lactone compounds, (S,R)-7, and its novel hydroxyamide precursors, (S,R)-6, is the following:

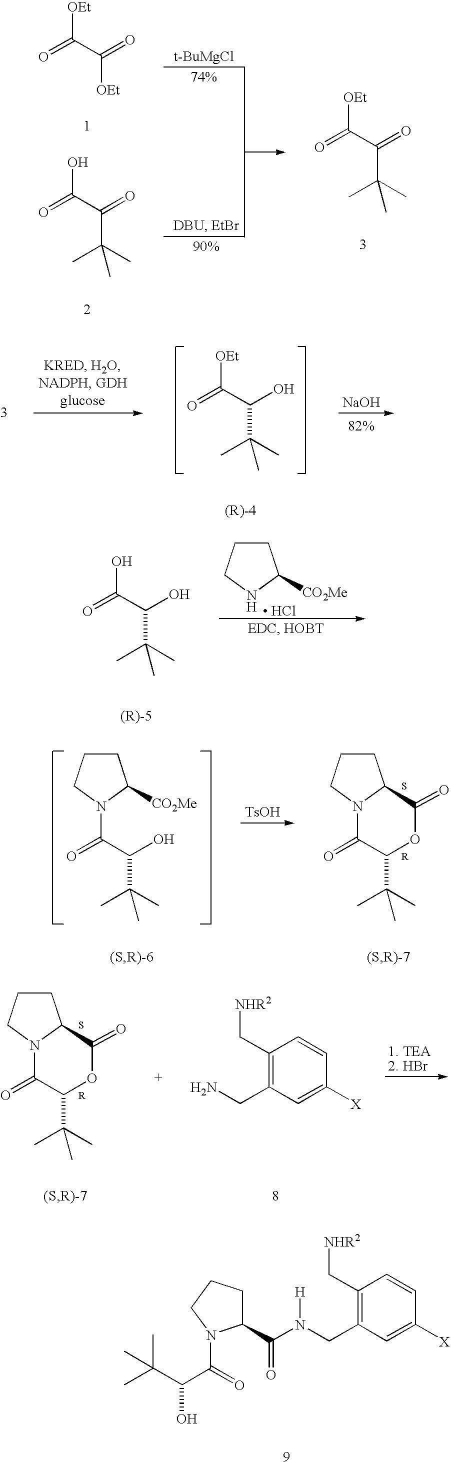

R² and X are as defined above.

Synthesis of N-heterocyclic Bicyclic Lactones

Step 1: Synthesis of (R)-2-hydroxy-3,3-dimethylbutanoic acid (R)-5

Ethyl 3,3-dimethyl-2-oxobutanoate 3 was synthesized either by the addition of t-BuMgCl to diethyl oxalate 1 or by the alkylation of 3,3-dimethyl-2-oxobutanoic acid 2 with EtBr/DBU. Rambaud, M.; Bakasse, M.; Duguay, G.; Villieras, J. SYNTHESIS 1988, 56; Kovács, L. Recl. Trav. Chim. Pays-Bas 1993, 112, 471; b) Cooper, A. J. L.; Ginos, J. Z, 2401.; Meister, A. Chem. Rev. 1983, 83, 321; Ono, N.; Yamada, T.; Saito, T.; Tanada, K.; Kaji, A. Bull. Chem. Soc. Jpn. 1978, 51, 2401. Both methods rapidly afforded keto ester 3 in high yields.

The first method of synthesizing Ethyl 3,3-dimethyl-2-oxobutanoate 3 is the following: To a solution of 498 g of diethyl oxalate in 2 L of toluene at −78° C. was added t-butyl magnesium chloride (1 M in THF) (3.85 L) over 1 h while maintaining the internal keep temperature at <−60° C. After 1 h, the reaction mixture was quenched with 3 N HCL (1 L) and water (1 L) and allowed to warm to room temperature. The organic phase was separated and washed with water. The solvent was removed in vacuo to yield 598.6 g of crude ketoester, which was purified by vacuum distillation (90° C., 40 mm Hg), to produce 443.3 g (74% yield) of ketoester (90 LCWP, 73 LCAP, 95 wt % by ¹H NMR).

The second method of synthesizing ethyl 3,3-dimethyl-2-oxobutanoate 3 is the following: To a mixture of 3,3-dimethyl-2-oxobutanoic acid 2 (35 g, 83 wt % by HPLC) and 260 mL of MTBE was slowly added of DBU(46.5 g). The addition was slightly exothermic. The reaction was cooled to 37° C. and of bromoethane (57 g) was added and stirred for 24 h at 38° C. The reaction mixture was washed with 1 N HCl, 10 wt % aq. NaCl, dried over Na₂SO₄ and concentrated in vacuo to afford 37.0 g of ketoester 3 as a light yellow oil [97% corrected yield (93 wt % by ¹H NMR, 92.5 LCWP), 89 LCAP). bp 90° C. (40 mm Hg); ¹H NMR (300 MHz, CDCl₃) Λ 4.30 (q, J=7.2 Hz, 2H), 1.34 (d, J=7.2 Hz, 3H), 1.24 (s, 9H); ¹³C NMR (75.5 MHz, CDCl₃) Λ 202.1, 163.9, 61.7, 42.6, 25.7 (3C), 14.1; IR (thin film) 2976, 1738 cm⁻¹, Anal Calcd. for C₈H₁₄O₃: C, 60.74; H, 8.92. Found: C, 60.79; H, 8.79.

The asymmetric reduction of 3 was accomplished with the isolated ketoreductase enzyme KRED 1001. KRED 1001 has been used to asymmetrically reduce Θ-ketoesters KRED1001 and is commercially available from BioCatalytics, Pasadena, Calif. 91106. The reduction is cofactor dependent. Typical operating parameters utilized ketoester 3 (45 mg/mL), NADPH (0.1-0.5 mg/mL), glucose dehydrogenase (0.5-2 mg/mL), KRED1001 (0.1-2 mg/mL) and glucose (70 mg/mL) in an appropriate buffer solution (e.g. phosphate, MOPS, etc.) at 25 to 40° C. with the pH being maintained between 6 and 8. The source of hydride for this ketone reduction was glucose. As monitored by HPLC, the conversion is quantitative and occurs with an extremely high degree of stereoselectivity (>500:1; R:S). The hydroxy ester (R)-4 could be isolated as an oil and then saponified to the corresponding enantiomerically pure hydroxy acid (R)-5 without epimerization. But in a more direct process, a simple pH adjustment (pH>13) of the crude enzymatic reaction mixture (post-reduction) immediately saponified the ethyl ester (R)-4. After pH adjustment (ca. pH=2), the desired product (R)-5 was extracted into ethyl acetate and the KRED, GDH, NADPH and glucose remained in the aqueous phase. Removal of the EtOAc and crystallization from heptane afforded the enantiomerically pure (R)-3,3-dimethyl-2-hydroxybutyric acid (R)-5 in an 82% isolated yield (>99.5% ee) and was successfully demonstrated on a 200 g scale.

The following components were rapidly stirred at room temperature: 4.6 L of 400 mM phosphate buffer stock solution, 12 L of the glucose stock solution, 320 mL of the GDH stock solution, 73.6 mL of the NADP stock solution, 12.8 mL of the KRED1001 stock solution and 295.2 g of keto ester 3. The pH was maintained at 7.0 by the addition of 5% NaOH during the course of the reaction. [The enantiomerically pure ethyl ester (S)-4 could be isolated at this stage, although the saponification reaction was most frequently performed on the crude reaction mixture: ¹H NMR (300 MHz, CDCl₃) Λ 4.33-4.21 (m, 2H), 3.80 (d, J=7.6 Hz, 1H), 2.83 (dd, J=7.6, 1.4 Hz, 1H), 1.32 (t, J=7.2 Hz, 3H), 0.981 (s, 9H); ¹³C NMR (75.5 MHz, CDCl₃) Λ 174.3, 78.4, 61.2, 35.2, 25.8, 14.2; IR (neat) 3515, 2960, 1727 cm⁻¹, [I]₃₆₅²⁵ −84 (c 1, MeOH); >95% ee] After complete reduction (8h, HPLC analysis), 720 mL of NaOH (50% v/v) was added and stirred for 75 min to effect complete saponification. The final hydrolyzed solution was neutralized to pH=2 with concentrated H₂SO₄ and then extracted with EtOAc. The solvent was removed in vacuo and the residue crystallized from heptane to afford 212.7 g of (R)-1-hydroxy-2,2-dimethylbutanoic acid (R)-5 as a white solid (86% yield, >99.5% ee): mp 49.8° C. (lit. mp 51° C.); [I]₃₆₅²⁵ −46 (c 1, MeOH); ¹H NMR (300 MHz, CDCl₃) Λ 3.93 (s, 1H), 1,04 (s, 9H); ¹³C NMR (75.5 MHz, CDCl₃) Λ 178.7, 78.3, 35.2, 25.8 (3C); IR (nujol mull) 3356, 2918, 2853, 1733 cm⁻¹, Anal Calcd for C₆H₁₂O₃: C, 54.53; H, 9.15. Found: C, 54.16; H, 9.09.

Step 2: Coupling Hydroxy Acid (R)-5 with an Ester

Amide formation between the enantiomerically pure hydroxy acid and L-proline methyl ester (EDC, HOBT, CH₃CN) afforded a mixture of the hydroxy ester (S,R)-6 and the lactone (S,R)-7 after aqueous workup. Treatment of the mixture with catalytic TsOH in toluene with the concomitant removal of MeOH from the system afforded exclusively the lactone (S,R)-7, which was crystallized from heptane. This procedure was demonstrated on >200 g scale to provide lactone (S,R)-7 as a white crystalline solid (73% isolated yield, >99LCWP, >99.5% de).

A solution of L-proline methyl ester hydrochloride (272.1 g, 1.64 mol) in 3 L of acetonitrile was cooled to 0° C. and diisopropylethylamine (215.6 gm, 1.67 equiv.) was added. After 15 minutes, HOBT (61.5 gm, 0.45 mole), hydroxyacid (R)-5 (200.1 gm, 1.51 mole) and EDC (350.9 gm, 1.83 mole) were added sequentially and the resulting mixture stirred at 0° C. for 5 h (90% HPLC assay yield). The mixture was quenched with 1 L of 3 N HCl and diluted with dichloromethane (3 L). The organic portion was separated and washed with 3 N HCl, saturated NaHCO₃ and then 10 wt % aqueous NaCl. The solvent was removed in vacuo to yield a crude mixture of ester and lactone (376.5 gm). This solid was dissolved in toluene (2 L) and placed in a 5 L 3-neck round-bottomed flask that was equipped with a short-path distillation head. To this was added PTSA (56.0 gm, 0.30 mole) and heated to 45° C. at 100 mm Hg. After 6 h at 45° C., the conversion to lactone was complete (100% by HPLC). During the distillation, approximately 500 mL of toluene was removed. The mixture was cooled to ambient temperature, washed twice with saturated NaHCO₃, saturated NaCl, dried over Na₂SO₄ and the solvent was removed in vacuo. The residue was recrystallized from heptane to afford 213.2 g of lactone (S,R)-7 as a white solid. The cyclization of hydroxy ester (S,R)-6 to lactone (S,R)-7 occurred rapidly. mp 111.6° C.; [α]₃₆₅²⁵ −214 (c 1, MeOH); ¹H NMR (300 MHz, CDCl₃) δ4.51 (s, 1H), 4.22 (dd, J=9.6,6.4 Hz, 1 H), 3.80-3.68 (m, 1H), 3.59-3.52 (m, 1H), 2.54-2.42 (m, 1H), 2.11-1.86 (m, 3H), 1.12 (s, 9H); ¹³C NMR (75.5 MHz, CDCl₃) δ167.8, 163.1, 89.6, 57.2, 45.8, 37.4, 30.5, 26.2 (3C), 21.9; IR (nujol mull) 2922, 2853, 1743, 1673 cm⁻¹, Anal Calcd. for C₁₁H₁₇NO₃: C, 62.54; H, 8.11; N, 6.63. Found: C, 62.57; H, 8.27; N, 6.51.

The following general methods were used in the above experiments: Elemental Analyses were performed by QTI, Whitehouse, NJ 08888. HPLC chromatograms were obtained on an Agilent model 1100 instrument with diode array detector. GC chromatograms were obtained on an Agilent 6850 series GC system. NMR spectra were obtained on Bruker spectrometer operating at 300 MHz for ¹H and at 75 MHz for ¹³C. The phosphate buffer stock solution was prepared as follows: 272.3 g of K₂HPO₄ was diluted with water to a final volume of 10 L while maintaining the pH=7 with 50% v/v NaOH. This 200 mM phosphate buffer solution was used to prepare the subsequent stock solutions: NADP solution (837 mg in 84 mL of 200 phosphate buffer solution); glucose solution (800 g in 2 L of phosphate buffer solution); GDH (3.51 g in 350 mL of 200 phosphate buffer solution); KRED-1001 (750 mg in 15 mL of buffer solution). LCAP refers to HPLC area percent and LCWP refers to HPLC weight percent. Racemic 4 was prepared by NaBH₄ reduction of ketoester 3 for chiral GC development: determined by chiral GC: CyclosilB, 30 m×0.25 mm ID×0.25 mm film thickness, isothermal 80° C., 1 TL/min He. Racemic hydroxy acid 5 was prepared by the saponification of racemic hydroxy ester 4 for chiral HPLC development: ChiralPak AD-H, 150 mm×4.6 mm; 85/15/0.1 hexane/EtOH/TFA, flow rate 1.5 TL/min. Methyl-2-bromo-5-chlorobenzoate was purchased from Esprit Chemical Co., Sarasota, Fla. 34243. KRED1001 was purchased from BioCatalytics, Pasadena, Calif. 91106. 3,3-Dimethyl-2-oxobutanoic acid was purchased from either Polycarbon Inc., Leominster, Mass. 01453 or Alfa Aesar, Ward Hill, Mass. 01835.

Step 3: Lactone Amination by Coupling (S,R)-7 Lactone with Amine 8

The coupling of the lactone (S,R)-7 with 8 (the preparation of which is described in WO 02/50056 on page 36) was accomplished in either TEA or IPA and occurred without the need for a catalyst or base additive. While many lactone aminolysis reactions require more rigorous conditions or are facilitated by the addition of catalysts, the facile nature of this amidation can be attributed to the inherent strain in lactone (S,R)-7.

Other polar solvents screened (NMP, DMF, DIPEA, CH₃CN, THF) resulted in slightly slower coupling rates or incompatibility with the lactone (MeOH, EtOH). The invention encompasses coupling the lactone with TEA or EPA or other polar solvents, such as NMP, DMF, DIPEA, CH₃CN, THF, as well as any other suitable reagents.

Alternatively, the lactone opening in THF could be accelerated by performing the reaction at 40° C. in the presence of HOAc. Regardless of the method for coupling the two fragments, the subsequent workup may include a wash with 2 M citric acid or similar reagent to remove amine 8 followed by a wash with 0.2 N NaOH or similar reagent (or saturated Na₂CO₃) to hydrolyze and remove unreacted lactone (S,R)-7. This process occurred without epimerization of either stereocenter. (S,R)-9 could be isolated as an amorphous solid following solvent removal, but this compound was typically utilized in the deprotection without isolation. Numerous conditions were screened for the unmasking of the benzyl amine by amino protecting group deprotection. Problems ranged from sluggish reactivity to product decomposition (spontaneous lactonization with extrusion of the corresponding diamine). The optimized conditions for amino protecting group removal incorporated the addition of a 6 wt % HBr (3 eq) solution to an anhydrous IPAc solution of the substrate. This afforded the target molecule HBr salt as a white amorphous solid in an overall 80% isolated yield from lactone (S,R)-7.

The above embodiment of the synthesis of novel N-heterocyclic bicyclic lactone compound is for illustration and not limitation. For example, a variety of hydroxy acids of formula (II) may be peptide coupled with a variety of esters of formula (III) using a variety of peptide coupling reagents to produce a variety of the novel hydroxy amides of formula (IV). The novel hydroxy amide of formula (IV) may be cyclized to the novel lactone of formula (I) using a variety of acids. Although lactone (S,R)-7, was coupled with compound 8 using TEA or IPA or other polar solvents, such as NMP, DMF, DIPEA, CH₃CN, THF, to create compounds of formula (V), a variety of solvents may be used to couple lactone (S,R)-7, with compound 8 to yield a variety of compounds of formula (V). The disclosed reagents, such as acids, peptide coupling reagents, and solvents, as well as amount of disclosed compounds may be modified by one of ordinary skill in the art without undue experimentation. The invention encompasses modification of the given ingredients, reagents, and ranges.

It will be apparent to those skilled in the art that various modifications and variations can be made to the novel N-heterocyclic bicyclic lactone compounds and its novel hydroxyamide precursors and synthesis of such N-heterocyclic bicyclic lactone compounds of the present invention without departing from the scope of the invention. Thus, the present invention include modifications and variations that are within the scope of the claims and their equivalents.

What is claimed is:

1. A compound of formula I or a pharmaceutically acceptable salt thereof, 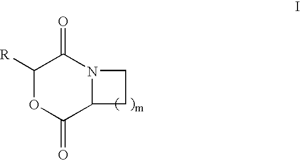
R is
a) C₁₋₆ alkyl unsubstituted or substituted with one, two, or three groups independently selected from C₆₋₁₀ aryl, C₁₋₆ alkoxy, halogen, and amino; or
b) a 6-10 membered monocyclic or bicyclic aryl ring system, unsubstituted or substituted with one, two or three groups independently selected from C₁₋₆ alkyl, C₁₋₆ alkoxy, halogen, and amino; and
m is 1, 2, 3, 4, or 5.

2. The compound of claim 1 wherein R is unsubstituted C₁₋₆ alkyl.

3. The compound of claim 2 wherein R is tert butyl.

4. The compound of claim 1 wherein m is 1.

5. A compound of formula IV or a pharmaceutically acceptable salt thereof, 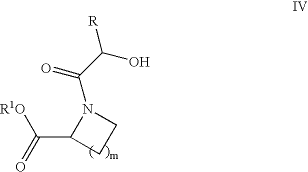
R is
a) C₁₋₆ alkyl unsubstituted or substituted with one, two, or three groups independently selected from C₆₋₁₀ aryl, C₁₋₆ alkoxy, halogen, and amino; or
b) a 6-10 membered monocyclic or bicyclic aryl ring system, unsubstituted or substituted with one, two or three groups independently selected from C₁₋₆ alkyl, C₁₋₆ alkoxy, halogen, and amino;
R¹ is
a) C₁₋₆ alkyl unsubstituted or substituted with one, two, or three groups independently selected from C₆₋₁₀ aryl, hydroxy, C₁₋₆ alkoxy, halogen, and amino;
b) benzyl unsubstituted or substituted with one, two or three groups independently selected from C₁₋₆ alkyl, hydroxy, C₁₋₆ alkoxy, halogen, and amino; or
c) hydrogen; and
m is 1, 2, 3, 4, or 5.

6. The compound of claim 5 wherein R is unsubstituted C₁₋₆ alkyl.

7. The compound of claim 6 wherein R is tert butyl and m is 1.

8. The compound of claim 5 wherein R¹ is methyl and m is 1.

9. A process of preparing a compound of formula I or a pharmaceutically acceptable salt thereof, 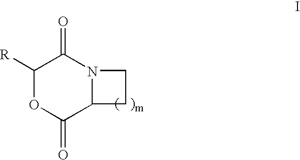
wherein
R is
a) C₁₋₆ alkyl unsubstituted or substituted with one, two, or three groups independently selected from C₆₋₁₀ aryl, C₁₋₆ alkoxy, halogen, and amino; or
b) a 6-10 membered monocyclic or bicyclic aryl ring system, unsubstituted or substituted with one, two or three groups independently selected from C₁₋₆ alkyl, C₁₋₆ alkoxy, halogen, and amino; and
m is 1, 2, 3, 4, or 5, comprising
1) coupling a hydroxy acid of formula II 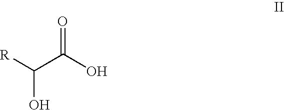
in presence of a peptide coupling reagent with a compound of formula III or a pharmaceutically acceptable salt thereof, 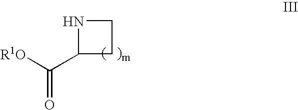
wherein
R¹ is
a) C₁₋₆ alkyl unsubstituted or substituted with one, two, or three groups independently selected from C₆₋₁₀ aryl, hydroxy, C₁₋₆ alkoxy, halogen, and amino;
b) benzyl unsubstituted or substituted with one, two or three groups independently selected from C₁₋₆ alkyl, hydroxy, C₁₋₆ alkoxy, halogen, and amino; or
c) hydrogen;
to produce a hydroxyamide of formula IV; 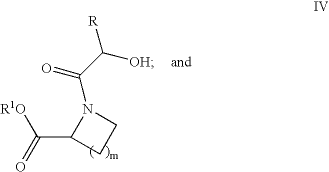
2) cyclizing the hydroxyamide of formula IV in the presence of an acid to produce formula I.

10. The process of claim 9 wherein R is unsubstituted C₁₋₆ alkyl.

11. The process of claim 10 wherein R is tert butyl and m=1.

12. The process of claim 9 wherein R¹ is methyl and m is 1.

13. The process of claim 9 wherein the peptide coupling reagent is 1-ethyl-3-(3-dimethylaminopropyl)carbodiimide hydrochloride.

14. The process of claim 9 wherein the acid is toluene sulfonic acid.

15. A process of preparing a compound of general formula V 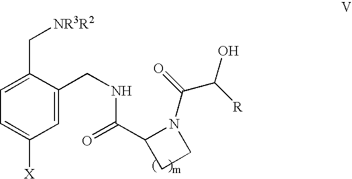
wherein
R is
a) C₁₋₆ alkyl unsubstituted or substituted with one, two, or three groups independently selected from C₆₋₁₀ aryl, C₁₋₆ alkoxy, halogen, and amino; or
b) 6-10 membered monocyclic or bicyclic aryl, unsubstituted or substituted with one, two or three groups independently selected from C₁₋₆ alkyl, C₁₋₆ alkoxy, halogen, and amino group;
R² is an amino protecting group;
m, is 1, 2, 3, 4, or 5; and
X is a halogen selected from the group consisting of F, Br, I, or Cl; comprising
1) coupling a compound of formula I, 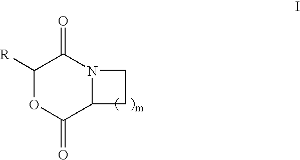
in the presence of a solvent, with a compound of the general formula 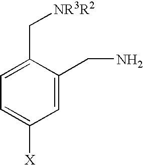
to form a compound of formula V.

16. The process of claim 15 wherein R² is tert butoxy carbonyl or carbobenzoxy.

17. The process of claim 15 wherein R is unsubstituted C₁₋₆ alkyl.

18. The process of claim 17 wherein R is tert butyl.

19. The process of claim 15 wherein the solvent is a polar solvent selected from the group of consisting of triethylamine, isopropyl alcohol, N-methyl pyrrolidinone, dimethylformamide, diisopropyethylamine, CH₃CN, and tetrahydrofuran.

20. The process of claim 15 wherein R³ is hydrogen.

21. A process of preparing a compound of formula IV or a pharmaceutically acceptable salt thereof, 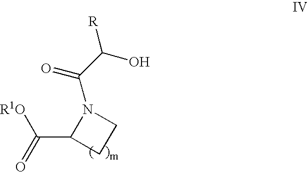
wherein
R is
a) C₁₋₆ alkyl unsubstituted or substituted with one, two, or three groups independently selected from C₆₋₁₀ aryl, C₁₋₆ alkoxy, halogen, and amino; or
b) a 6-10 membered monocyclic or bicyclic aryl ring system, unsubstituted or substituted with one, two or three groups independently selected from C₁₋₆ alkyl, C₁₋₆ alkoxy, halogen, and amino; and
m is 1, 2, 3, 4, or 5; and
R¹ is
a) C₁₋₆ alkyl unsubstituted or substituted with one, two, or three groups independently selected from C₆₋₁₀ aryl, hydroxy, C₁₋₆ alkoxy, halogen, and amino;
b) benzyl unsubstituted or substituted with one, two or three groups independently selected from C₁₋₆ alkyl, hydroxy, C₁₋₆ alkoxy, halogen, and amino; or
c) hydrogen;
comprising coupling a hydroxy acid of formula II 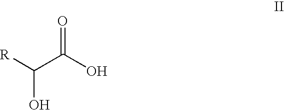
in presence of a peptide coupling reagent, with a compound of formula III or a pharmaceutically: acceptable salt thereof, 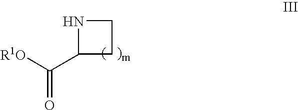
to produce a compound of formula IV.
